(19) United States
(12) Patent Application Publication
(10) Pub. No.: US 2026/0242710 A1
(43) Pub. Date: Aug. 20, 2026

(54) A MICROFLUIDIC DEVICE AND A METHOD OF FORMING THEREOF

(71) Applicants: The University of Sydney, NSW (AU); The Heart Research Institute Limited, NSW (AU)

(72) Inventors: Yunduo Zhao, Sydney (AU); Zihao Wang, Sydney (AU); Lining Arnold Ju, Sydney (AU); Yingqi Zhang, Sydney (AU)

(21) Appl. No.: 19/162,050

(22) PCT Filed: Mar. 6, 2024
(86) PCT No.: PCT/AU2024/050185

§ 371 Date: Sep. 3, 2025

(30) Foreign Application Priority Data
Mar. 6, 2023 (AU) 2023900588

(51) Int. Cl. C12M 3/06; A61B 5/02; A61B 5/055; A61B 6/03; B01L 3/00; C12M 3/00

(52) U.S. Cl. CPC C12M 23/16; A61B 5/02007; A61B 5/055; A61B 6/03; B01L 3/502707; C12M 21/08; B01L 2200/027; B01L 2200/12

(57) ABSTRACT

A method of manufacturing a patient-specific microfluidic device and a patient-specific microfluidic device are provided. The method includes obtaining three-dimensional (3D) information associated with vascular geometry in a region of interest from one or more clinical images associated with a patient, and fabricating the patient-specific microfluidic device using said 3D information. The patient-specific microfluidic device includes a flow channel having a geometry that substantially corresponds to the vascular geometry in the region of interest.

### FIELD OF THE INVENTION

The present invention generally relates to microfluidic devices and methods of forming such devices. In particular, the invention relates to microfluidic devices adapted to be patient-specific.

### BACKGROUND OF THE INVENTION

Blood clot (thrombosis) triggered cardiovascular diseases such as heart attack and stroke are the number one killer worldwide, resulting in an average of two people dying every hour in Australia. Thrombotic risk assessment generally relies on invasive digital subtraction angiography—insertion of a catheter into an artery and passing it up to the blood vessels—despite recent advances in clinical imaging modalities. Although there have been recent developments in 4D MRI and phase-contrast technologies, it is still not possible to accurately quantify ‘Virchow's triad’ (relating to factors that contribute to thrombosis formation) in the cardiovascular or cerebral vasculature.

Blood coagulation tests and platelet function analyses have previously been undertaken to predict thrombotic risk. However, such methodologies are reliant on labour-intensive testing, bulky and costly equipment, and specialist interpretation—all of which are poorly available or difficult to access in regional and rural health settings.

Engineered tissue systems, such as bioreactors and vascular network devices, have been developed in the past to mimic various biological conditions in vitro in order to help health professionals study and test the efficacy of certain drugs before formal administration en masse. This includes culturing various types of cells, and subsequently conducting experiments on the cultured cells.

Microfluidics based bioreactors and vascular network mimicking microchannels have been utilised in various forms for use in drug discovery and development, as they can provide more accurate and physiologically relevant model systems for predicting and/or testing the pharmacokinetic and pharmacodynamic responses associated with pharmacologic agents. Among the major challenges of such devices is the need for more complex and physiologically relevant systems that better mimic the structure, physiology, and function, of native biological tissues. This is particularly important and challenging when attempting to use engineered tissues to screen, test, and/or evaluate therapeutic agents. Vascular network microdevices are often relatively standardised devices, and can be expensive and timely to produce, particularly at scale.

It is desirable for at least one embodiment of the invention to provide an improved device for predicting blood clot events for a patient. It may also be desirable for at least one embodiment of the invention to provide an improved method of manufacturing such devices. Alternatively, it is desirable for at least one embodiment of the invention to provide a useful alternative to known prior art devices and/or methods.

Reference to any prior art in the specification is not an acknowledgment or suggestion that this prior art forms part of the common general knowledge in any jurisdiction or that this prior art could reasonably be expected to be understood, regarded as relevant, and/or combined with other pieces of prior art by a skilled person in the art.

### SUMMARY OF THE INVENTION

In a first aspect, the present invention provides a method of manufacturing a patient-specific microfluidic device, the method including:
obtaining three-dimensional (3D) information associated with vascular geometry in a region of interest from one or more clinical images associated with a patient;
fabricating the patient-specific microfluidic device using said 3D information, wherein said patient-specific microfluidic device includes a flow channel having a geometry that substantially corresponds to the vascular geometry in the region of interest.

Advantageously, the present invention enables the manufacturing of a patient-specific microfluidic device that accurately reflects the vasculature of the patient in a particular region of interest. This is achieved by producing a flow channel in the microfluidic device based on 3D information derived from the one or more clinical images associated with the patient. Thus, the geometry of the flow channel substantially replicates the venous geometry of the patient in the particular region of interest. The patient-specific nature of the microfluidic device enables more reliable information to be gathered about the patient that enables improved prediction of a patient's pro-thrombotic tendency (and thus potentially preventing life-threatening events such as strokes or heart attacks). For example, the patient-specific microfluidic device enables assessment of the patient's Virchow's triad, which can provide valuable information for clinicians in terms of more targeted and effective treatments, and even personalised drug efficiency. The microfluidic device can also be used for short to long term monitoring of cardiovascular patients with conditions such as stroke, coronary artery stenosis, arteriovenous malformation, and aneurysm.

A further advantage of the present invention is it can enable inexpensive manufacturing of a patient-specific microfluidic device, including in some cases when manufactured at scale.

A further advantage of the present invention is it enables accurate microfluidic devices to be manufactured without the requirement for core facility cleanrooms (that includes staff support) that can be costly and result in slow turnover times. Further, the invention lends itself to scaled up manufacturing, whereby a plurality of microfluidic devices can be readily manufactured.

Preferably, the microfluidic device is in the form of a microvessel-on-chip device.

In an embodiment, the one or more clinical images include one or more of magnetic resonance imaging (MRI) scans (such as magnetic resonance venography (MRV) scans), computerised tomography (CT) scans, and any other clinical images that contain 3D anatomical information. In an embodiment, the one or more clinical images include one or more 2D scans, wherein said 3D information can be obtained or inferred from said one or more 2D scans.

In an embodiment, the method further includes obtaining said one or more clinical images associated with the patient.

In an embodiment, said obtaining three-dimensional (3D) information includes identifying the region of interest from the one or more clinical images, and converting the one or more clinical images into 3D vascular contours at least in the region of interest. The 3D vascular contours may be generated from a plurality of scatter points associated with the vascular geometry in the region of interest. In an embodiment, the 3D vascular contours include continuous surfaces constructed from the scatter points, thereby producing surface profiles of the vascular geometry in the region of interest. In an embodiment, the surface profiles of the vascular geometry in the region of interest are smoothed using a computer algorithm (e.g. a surface smoothing algorithm).

In an embodiment, the method further includes fabricating a mould from said 3D information, wherein said mould includes geometry that substantially corresponds to the vascular geometry in the region of interest.

Preferably, fabricating a mould includes fabricating a first mould portion and a second mould portion, wherein said first mould portion includes geometry that substantially corresponds to a first portion of the vascular geometry in the region of interest, and said second mould portion includes geometry that substantially corresponds to a second portion of the vascular geometry in the region of interest. Preferably, the first portion and the second portion together define geometry that substantially corresponds to the entirety of the vascular geometry in the region of interest.

In an embodiment, said geometry of the first mould portion that substantially corresponds to the first portion of the vascular geometry in the region of interest is in the form of a recess extending from an inner surface of the first mould portion towards an outer surface of the first mould portion. In this way, the first mould portion is in the form of a negative mould. In an embodiment, said geometry of the second mould portion that substantially corresponds to the second portion of the vascular geometry in the region of interest is in the form of a recess extending from an inner surface of the second mould portion towards an outer surface of the second mould portion. In this way, the second mould portion is in the form of a negative mould. Each of the respective recesses of the first mould portion and the second mould portion may include a substantially hemispherical internal profile. However, it will be appreciated that the precise internal profile of the recesses will correspond to the respective first and second portions of the vascular geometry in the region of interest. Advantageously, by forming the first and second mould portions, each including geometry corresponding to a portion of the entirety of the vascular geometry in the region of interest separately and utilising these mould portions in the method of manufacturing the patient-specific microfluidic device, a greater representation of the complex shaping of the vasculature can be captured and replicated in the patient-specific microfluidic device.

In an embodiment, said fabricating the mould includes fabricating the mould by an additive manufacturing process. Preferably, the mould is fabricated using a stereolithography (SLA) 3D printing process. Advantageously, this can enable relatively inexpensive and rapid manufacture of the microfluidic devices.

In an embodiment, fabricating the first mould portion includes forming a groove extending from the inner surface of the first mould portion towards the outer surface of the first mould portion, wherein said groove substantially surrounds the recess. Preferably, said groove at least in part delineates a spacer that substantially surrounds the recess, wherein a height of said spacer corresponds to a depth of the groove and defines a portion of the first mould portion to be removed. The spacer may be delineated at least in part by said groove and an outer periphery of the first mould portion. In an embodiment, fabricating the second mould portion includes forming a groove extending from the inner surface of the second mould portion towards the outer surface of the second mould portion, wherein said groove substantially surrounds the recess. Preferably, said groove at least in part delineates a spacer that substantially surrounds the recess, wherein a height of said spacer corresponds to a depth of the groove and defines a portion of the second mould portion to be removed. The spacer may be delineated at least in part by said groove and an outer periphery of the second mould portion.

In an embodiment, the method further includes polishing the inner surface of the first mould portion and the second mould portion, wherein said polishing includes removing said portion of each of the first and second mould portions, thereby forming a polished inner surface of the first and second mould portions. Preferably, polishing the inner surface of the first mould portion and the second mould portion includes a multi-stage polishing procedure, including a coarse polishing stage and a fine polishing stage.

In an embodiment, the method further includes fabricating a secondary mould from the mould, wherein said secondary mould includes geometry that substantially corresponds to the vascular geometry in the region of interest. Preferably, fabricating the secondary mould includes fabricating a first secondary mould portion and a second secondary mould portion, wherein said first secondary mould portion includes geometry that substantially corresponds to the first portion of the vascular geometry in the region of interest, and said second secondary mould portion includes geometry that substantially corresponds to the second portion of the vascular geometry in the region of interest.

In an embodiment, the method includes fabricating the first secondary mould from the first mould portion and fabricating the second secondary mould from the second mould portion. In an embodiment, said geometry of the first secondary mould portion that substantially corresponds to the first portion of the vascular geometry in the region of interest is in the form of a protrusion extending outwardly from an inner surface of the first secondary mould portion. In this way, the first secondary mould portion is in the form of a positive mould. In an embodiment, said geometry of the second secondary mould portion that substantially corresponds to the second portion of the vascular geometry in the region of interest is in the form of a protrusion extending outwardly from an inner surface of the second secondary mould portion. In this way, the second secondary mould portion is in the form of a positive mould. Each of the respective protrusions of the first secondary mould portion and the second secondary mould portion may include a substantially hemispherical external profile. However, it will be appreciated that the precise external profile of the protrusions will correspond to the respective first and second portions of the vascular geometry in the region of interest. Advantageously, by forming the first and second secondary mould portions, each including geometry corresponding to a portion of the entirety of the vascular geometry in the region of interest separately and utilising these mould portions in the method of manufacturing the patient-specific microfluidic device, a greater representation of the complex shaping of the vasculature can be captured and replicated in the patient-specific microfluidic device.

In an embodiment, the secondary mould is formed at least in part of polydimethylsiloxane (PDMS). In an embodiment, each of the first and second secondary mould portions are formed at least in part of PDMS. In an embodiment, the secondary mould is formed of a PDMS/curing agent mixture having a PDMS/curing agent ratio between about 1:4 to about 1:6 ratio. Preferably, the mixture includes a PDMS/curing agent ratio of about 1:5.

In an embodiment, the first secondary mould portion includes one or more moulding spacers projecting from an inner surface thereof. In an embodiment, the second secondary mould portion includes one or more moulding spacers projecting from an inner surface thereof. In an embodiment, each of the first and second secondary mould portions include one or more moulding spacers projecting from the inner surface thereof. Preferably, the one or more moulding spacers are configured to support a glass cover slide in spaced relation from the inner surface of the respective first and/or second secondary mould portion. In an embodiment, the one or more moulding spacers are integrally formed with their respective secondary mould portion. Alternatively, the one or more moulding spacers are affixed to the respective secondary mould portions after the secondary mould portions are formed. Preferably, the moulding spacers have a length of about 170 μm from the inner surface of the respective first and/or second secondary mould portions. In an embodiment, the one or more moulding spacers have a rounded external profile. For example, the one or more moulding spacers may be of hemispherical or cylindrical form. In alternative embodiments, the one or more moulding spacers have an external profile of different from, such as rectangular.

In an embodiment, fabricating the patient-specific microfluidic device includes forming the patient-specific microfluidic device from the secondary mould, wherein said patient-specific microfluidic device includes geometry that substantially corresponds to the vascular geometry in the region of interest. Preferably, the patient-specific microfluidic device includes a first patient-specific microfluidic device portion and a second patient-specific microfluidic device portion, wherein said first patient-specific microfluidic device portion includes geometry that substantially corresponds to the first portion of the vascular geometry in the region of interest, and said second patient-specific microfluidic device portion includes geometry that substantially corresponds to the second portion of the vascular geometry in the region of interest. In an embodiment, said geometry of the first patient-specific microfluidic device portion that substantially corresponds to the first portion of the vascular geometry in the region of interest is in the form of a recess extending from an inner surface of the first patient-specific microfluidic device portion towards an outer surface of the first patient-specific microfluidic device portion. In an embodiment, said geometry of the second patient-specific microfluidic device portion that substantially corresponds to the second portion of the vascular geometry in the region of interest is in the form of a recess extending from an inner surface of the second patient-specific microfluidic device portion to an outer surface of the second patient-specific microfluidic device portion.

Preferably, the method includes fabricating the first patient-specific microfluidic device portion from the first secondary mould portion and forming the second patient-specific microfluidic device portion from the second secondary mould portion. In an embodiment, said forming the first patient-specific microfluidic device portion includes positioning a glass cover slide on the one or more moulding spacers of the first secondary mould portion such that the glass cover is in spaced relation with the inner surface of the first secondary mould portion, wherein a moulding chamber is formed between the glass cover and the inner surface of the first secondary mould portion. The method may further include injecting a settable material into the moulding chamber, allowing the settable material to cure, thereby forming said first patient-specific microfluidic device portion. In an embodiment, said forming the second patient-specific microfluidic device portion includes positioning a glass cover slide on the one or more moulding spacers of the second secondary mould portion such that the glass cover is in spaced relation with the inner surface of the second secondary mould portion, wherein a moulding chamber is formed between the glass cover and the inner surface of the second secondary mould portion. The method may further include injecting a settable material into the moulding chamber, allowing the settable material to cure, thereby forming said second patient-specific microfluidic device portion.

The settable material may be a PDMS/curing agent mixture. In an embodiment, the PDMS/curing agent mixture has a PDMS/curing agent ratio between about 1:9 to about 1:11. Preferably, the mixture includes a PDMS/curing agent ratio of about 1:10.

In an embodiment, the method further includes forming an inlet channel and an outlet channel in the patient-specific microfluidic device, wherein the inlet channel and the outlet channel are in fluid communication with the flow channel. Preferably, the inlet channel and the outlet channel are formed in one of the first patient-specific microfluidic device portion or the second patient-specific microfluidic device portion. Preferably, the inlet channel is in fluid communication with an upstream end of the flow channel, and the outlet channel is in fluid communication with a downstream end of the flow channel.

In an embodiment, the method further includes subjecting the patient-specific microfluidic device to a plasma cleaning process. Preferably, the method includes subjecting the first patient-specific microfluidic device portion and the second patient-specific microfluidic device portion to a plasma cleaning process.

In an embodiment, the method further includes joining the first patient-specific microfluidic device portion to the second patient-specific microfluidic device portion along the respective inner surfaces thereof, such that the recess of each respective microfluidic device portion substantially overlap along their longitudinal extent, thereby forming said flow channel having the geometry that substantially corresponds to the vascular geometry in the region of interest. In an embodiment, said joining includes bonding. Preferably, said bonding is a covalent bond. In an alternative embodiment, said bonding the first patient-specific microfluidic device portion to the second patient-specific microfluidic device portion includes utilising an ultraviolet (UV) cured adhesive. In one embodiment, the inner surfaces of the first and second patient-specific microfluidic device portions are filled with the UV cured adhesive and the first and second patient-specific microfluidic device portions are then carefully aligned under a microscope. This may then be followed by flowing water at, for example, 80° C. through the flow channel to remove any excess adhesive within the flow channel, while maintaining contact between the inner surfaces of the first and second patient-specific microfluidic device portions. In an optional subsequent step, the first and second patient-specific microfluidic device portions are exposed to UV light to cure the adhesive and thereby bond the first and second patient-specific microfluidic device portions together. In an embodiment, said joining includes mechanically fastening the first patient-specific microfluidic device portion to the second patient-specific microfluidic device portion. For example, the first patient-specific microfluidic device portion to the second patient-specific microfluidic device portion may be clamped to one another.

In an embodiment, fabricating the patient-specific microfluidic device includes fabricating the patient-specific microfluidic device in a two stage moulding process, wherein the first stage of the moulding process includes fabricating said secondary mould from the mould, and wherein the second stage of the moulding process includes said fabricating the patient-specific microfluidic device from the secondary mould.

In an embodiment, the method further includes endothelialising the patient-specific microfluidic device.

In an embodiment, said method of manufacturing a patient-specific microfluidic device includes manufacturing a plurality of patient-specific microfluidic devices, including obtaining three-dimensional (3D) information associated with vascular geometry in a region of interest from one or more clinical images for each respective patient of a plurality of patients; fabricating the plurality of patient-specific microfluidic devices using said 3D information for each respective patient, wherein said plurality of patient-specific microfluidic devices each include a flow channel having a geometry that substantially corresponds to the vascular geometry in the region of interest for each respective patient.

In an embodiment, said method of manufacturing a patient-specific microfluidic device includes manufacturing a plurality of patient-specific microfluidic devices, including obtaining three-dimensional (3D) information associated with vascular geometry in a plurality of regions of interest from one or more clinical images associated with a patient; fabricating the plurality of patient-specific microfluidic devices using said 3D information, wherein said plurality of patient-specific microfluidic devices each include a flow channel having a geometry that substantially corresponds to the vascular geometry in the respective regions of interest.

Advantageously, such embodiments allow scale up in manufacture by enabling a plurality of microfluidic devices to be produced by the method.

In a second aspect, the present invention provides a patient-specific microfluidic device manufactured using the method of the first aspect of the invention.

It will be appreciated that features disclosed with respect to the first aspect of the invention are also applicable with respect to the second aspect of the invention, including different combinations of features disclosed.

In a third aspect, the present invention provides a patient-specific microfluidic device, the device including a flow channel having a geometry that substantially corresponds to a patient's vascular geometry in a region of interest, said geometry derived from one or more clinical images associated with the patient.

Advantageously, the present invention provides a patient-specific microfluidic device that accurately reflects the vasculature of the patient in a particular region of interest. This is achieved by providing a flow channel in the microfluidic device having geometry based on information derived from one or more clinical images associated with the patient. Thus, the geometry of the flow channel substantially replicates the venous geometry of the patient in the particular region of interest. The patient-specific nature of the microfluidic device enables more reliable information to be gathered about the patient that enables improved prediction of a patient's pro-thrombotic tendency (and thus potentially preventing life-threatening events such as strokes or heart attacks). For example, the patient-specific microfluidic device enables assessment of the patient's Virchow's triad, which can provide valuable information for clinicians in terms of more targeted and effective treatments, and even personalised drug efficiency. The microfluidic device can also be used for short to long term monitoring of cardiovascular patients with conditions such as stroke, coronary artery stenosis, arteriovenous malformation, and aneurysm.

In an embodiment, the one or more clinical images include one or more magnetic resonance imaging (MRI) scans (such as magnetic resonance venography (MRV) scans), computerised tomography (CT) scans, and any other clinical images that contain 3D anatomical information. In an embodiment, the one or more clinical images include one or more 2D scans, wherein said 3D information can be obtained or inferred from said one or more 2D scans

In an embodiment, the patient-specific microfluidic device includes a first patient-specific microfluidic device portion and a second patient-specific microfluidic device portion joined to the first patient-specific microfluidic device portion, wherein said first patient-specific microfluidic device portion includes geometry that substantially corresponds to a first portion of the vascular geometry in the region of interest, and said second patient-specific microfluidic device portion includes geometry that substantially corresponds to a second portion of the vascular geometry in the region of interest. Preferably, the first portion and the second portion together define geometry that substantially corresponds to the entirety of the vascular geometry in the region of interest.

In an embodiment, said geometry of the first patient-specific microfluidic device portion that substantially corresponds to the first portion of the vascular geometry in the region of interest is in the form of a recess extending from an inner surface of the first patient-specific microfluidic device portion towards an outer surface of the first patient-specific microfluidic device portion. In an embodiment, said geometry of the second patient-specific microfluidic device portion that substantially corresponds to the second portion of the vascular geometry in the region of interest is in the form of a recess extending from an inner surface of the second patient-specific microfluidic device portion to an outer surface of the second patient-specific microfluidic device portion. Each of the respective recesses of the first patient-specific microfluidic device portion and the second patient-specific microfluidic device portion may include a substantially hemispherical internal profile. However, it will be appreciated that the precise internal profile of the recesses will correspond to the respective first and second portions of the vascular geometry in the region of interest. Advantageously, by separately forming the first and second patient-specific microfluidic device portions, each including geometry corresponding to a portion of the entirety of the vascular geometry in the region of interest, a greater representation of the complex shaping of the vasculature can be captured and replicated in the patient-specific microfluidic device.

In an embodiment, the first patient-specific microfluidic device portion and the second patient-specific microfluidic device portion are joined along respective inner surfaces thereof, such that the recess of each respective microfluidic device portion substantially overlap along their longitudinal extent, thereby forming said flow channel having the geometry that substantially corresponds to the vascular geometry in the region of interest

In an embodiment, the patient-specific microfluidic device is formed of a settable material. In an embodiment, each of the first and second patient-specific microfluidic device portions are formed of a settable material. The settable material may be a PDMS/curing agent mixture. In an embodiment, the PDMS/curing agent mixture has a PDMS/curing agent ratio between about 1:9 to about 1:11. Preferably, the mixture includes a PDMS/curing agent ratio of about 1:10.

In an embodiment, the patient-specific microfluidic device includes an inlet channel in fluid communication with an upstream end of the flow channel, and an outlet channel in fluid communication with a downstream end of the flow channel. Preferably, the inlet channel and the outlet channel are formed in one of the first patient-specific microfluidic device portion or the second patient-specific microfluidic device portion.

In an embodiment, the patient-specific microfluidic device includes endothelial tissue, said endothelial tissue covering at least a portion of the flow channel. In an embodiment, the endothelial tissue covers at least a portion of a wall, which at least in part defines said flow channel. Preferably, said endothelial tissue covers a substantial portion of the internal profile of the flow channel.

It will be appreciated that features disclosed with respect to the first and second aspects of the invention are also applicable with respect to the third aspect of the invention, including different combinations of features disclosed.

As used herein, except where the context requires otherwise, the term “comprise” and variations of the term, such as “comprising”, “comprises” and “comprised”, are not intended to exclude further additives, components, integers or steps.

Further aspects of the present invention and further embodiments of the aspects described in the preceding paragraphs will become apparent from the following description, given by way of example and with reference to the accompanying drawings.

### BRIEF DESCRIPTION OF THE DRAWINGS

FIG. 1 is a top view of a microfluidic device in accordance with an embodiment of the present invention;

FIG. 2 is a side view of the microfluidic device of FIG. 1;

FIG. 3 is a perspective view of a test kit including the microfluidic device of FIG. 1;

FIG. 4 is a flow diagram of a method of manufacturing a microfluidic device in accordance with an embodiment of the invention;

FIG. 5 illustrates MRI scans of a particular patient;

FIG. 6 illustrates an image processing step of the method of FIG. 4;

FIG. 7 illustrates a two-stage moulding process of the method of FIG. 4;

FIG. 8 is a top view of a positive mould PDMS chip used in the method of FIG. 4;

FIG. 9 is a front view of a positive mould PDMS chip during a second stage of the moulding process; and

FIG. 10 illustrates a method of manufacturing a plurality of microfluidic devices in accordance with an embodiment of the invention.

### DETAILED DESCRIPTION OF THE EMBODIMENTS

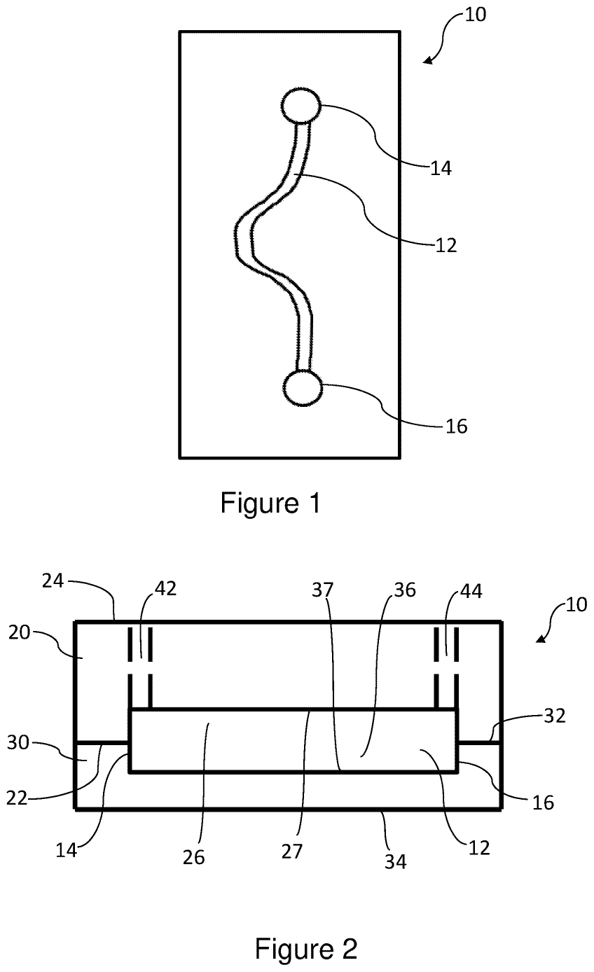

Reference is made to FIGS. 1 and 2, which illustrate a patient-specific microfluidic device in the form of a patient-specific microvessel-on-chip device 10. In the present embodiment, patient-specific microvessel-on-chip device 10 is a miniaturised and transparent microfluidic device that enables real-time visualisation of a coagulation process when a blood sample is moved through patient-specific flow channel 12, from an upstream end 14 thereof to a downstream end 16 thereof. As will become evident from the description below, microvessel-on-chip device 10 can provide a low-cost, personalised and scalable tool for thrombotic assessment.

Microvessel-on-chip device 10 provides an in-vitro assessment of whether a patient may be prone to suffering a blood clot at a particular location (i.e. at a particular diseased blood vessel) by mimicking the blood flow process through said location. The advantages of microvessel-on-chip device 10 is that it can help replicate the complex interplay between blood flow and blood clots under the flow disturbance that would be encountered at the location. To this end, microvessel-on-chip device 10 takes into account two particularly important characteristics in order to suitably model a possible blood clot event—(1) patient-specific vascular morphology, and (2) disturbed fluid dynamics.

As will be described in further detail below, the patient-specific vascular morphology provided by microvessel-on-chip device 10 is provided by fabricating microvessel-on-chip device 10 with patient-specific flow channel 12. Patient-specific flow channel 12 is a model of a particular blood vessel derived from one or more clinical images of the patient's vasculature. For example, one or more CT scans of the patient's cerebral vasculature may be utilised to produce microvessel-on-chip device 10 having patient-specific flow channel 12, wherein patient-specific flow channel 12 models, for example, the patient's superior sagittal sinus, sigmoid sinus, coronary artery or the carotid artery. Utilising an accurate representation of the patient's vasculature at a particular region of interest enables more accurate modelling of blood flow through the blood vessel. This is because patient-specific flow channel 12 will reflect any patient-specific blood vessel structure abnormalities (e.g. stenosis, bifurcation and aneurysm). Presence of such structural abnormalities will influence the fluid mechanics of blood flow through the blood vessel that would otherwise not be captured in some known prior art microfluidic devices. However, these fluid mechanic effects would be captured by microvessel-on-chip device 10. Therefore, microvessel-on-chip device 10 will provide more accurate modelling of blood flow through a particular blood vessel of a patient, and thereby enable better prediction of a possible blood clot event. Furthermore, patient-specific microfluidic device 10 can also be used for drug screening by moving a blood sample that includes a therapeutic dosage of a drug through flow channel 12. This allows for more accurate assessments of the efficacy of certain drugs for a particular patient, leading to more personalised and effective treatments.

As best shown in FIGS. 1 and 2, microvessel-on-chip device 10 is of substantially planar form. Microvessel-on-chip device 10 includes a first microvessel-on-chip device portion 20 and a second microvessel-on-chip device portion 30. First microvessel-on-chip device portion 20 is substantially in the shape of rectangular prism, having an inner surface 22 and an opposed outer surface 24. A recess 26 extends from inner surface 22 towards outer surface 24. Recess 26 includes a substantially hemispherical internal profile 27 when viewed in transverse cross-section, which defines a substantially hemispherical internal profile of patient-specific flow channel 12. It will be understood that whilst recess 26 is defined as having a substantially hemispherical internal profile 27, in actual fact internal profile 27 will have a more complex shape that corresponds to the internal profile of a portion of the blood vessel which is being modelled—in this case it will be an ‘upper portion’ of the blood vessel when viewed in the orientation shown in FIG. 2. As will be appreciated by a person skilled in the art, blood vessels do not have a constant cross-sectional area along their length and this will be reflected by recess 26, which is formed based on one or more clinical images of the patient's vasculature at the desired region of interest (e.g. a desired blood vessel).

Similarly, second microvessel-on-chip device portion 30 is substantially in the shape of rectangular prism, having an inner surface 32 and an opposed outer surface 34. A recess 36 extends from inner surface 32 towards outer surface 34. Recess 36 includes a substantially hemispherical internal profile 37 when viewed in transverse cross-section, which defines a substantially hemispherical internal profile of patient-specific flow channel 12. Again, it will be understood that whilst recess 36 is defined as having a substantially hemispherical internal profile 37, in actual fact internal profile 37 will have a more complex shape that corresponds to a portion of the external profile of the blood vessel which is being modelled—in this case it will be a ‘lower portion’ of the blood vessel when viewed in the orientation shown in FIG. 2. As previously mentioned, blood vessels do not necessarily have a constant cross-sectional area along their length and this will be reflected by recess 36.

It will be evident that rather than forming patient-specific flow channel 12 in microvessel-on-chip device 10 as a single part, patient-specific flow channel 12 has been formed in two parts—providing recess 26 on first microvessel-on-chip device portion 20, which provides a substantially hemispherical portion of the internal profile of the blood vessel, and providing recess 36 on second microvessel-on-chip device portion 30, which provides another substantially hemispherical portion of the internal profile of the blood vessel. Together, the two substantially hemispherical portions formed by recesses 26, 36 provide a substantially spherical internal profile of the blood vessel. Forming the substantially spherical internal profile of the blood vessel from two substantially hemispherical internal profiles is advantageous as it enables each substantially hemispherical internal profile to capture finer detail of the rather complex shaped internal profile of the blood vessel, thereby allowing more accurate modelling of the overall spherical internal profile of the blood vessel. In other words, forming the substantially spherical internal profile of the blood vessel in two separate parts enables more accurate fabrication of the complex anatomies of each individual substantially hemispherical internal profile and therefore a more accurate combined internal profile of patient-specific flow channel 12.

First microvessel-on-chip device portion 20 and second microvessel-on-chip device portion 30 are, in some embodiments, covalently bonded together such that respective recess 26, 36 of each microvessel-on-chip device portion substantially overlap along their longitudinal extent, thereby forming said patient-specific flow channel 12 having the internal profile that substantially corresponds to the vascular geometry in the region of interest.

Microvessel-on-chip device 10 includes an inlet channel 42 that extends from an outer surface of one of the first microvessel-on-chip device portion 20 or second microvessel-on-chip device portion 30 to an inner surface thereof such that inlet channel 42 is in fluid communication with upstream end 14 of patient-specific flow channel 12. In the depicted embodiment, inlet channel 42 is formed in first microvessel-on-chip device portion 20 and extends from outer surface 24 to inner surface 22. Microvessel-on-chip device 10 further includes an outlet channel 44 that extends from an outer surface of one of the first microvessel-on-chip device portion 20 or second microvessel-on-chip device portion 30 to an inner surface thereof such that outlet channel 44 is in fluid communication with downstream end 14 of patient-specific flow channel 12. In the depicted embodiment, outlet channel 44 is also formed in first microvessel-on-chip device portion 20 and extends from outer surface 24 to inner surface 22. It is preferred that both inlet and outlet channels be formed on the same microvessel-on-chip device portion, although this is not essential.

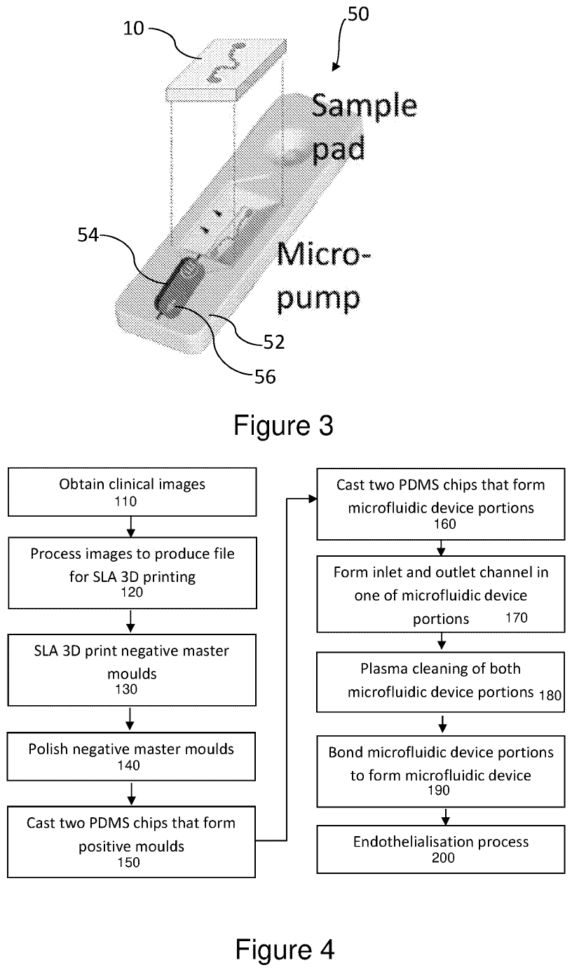

Inlet channel 42 is configured to receive a blood sample and convey the blood sample to patient-specific flow channel 12. In order to drive the blood sample through patient-specific flow channel 12, microvessel-on-chip device 10 is operatively engaged with a suitable pump 54 (FIG. 3) that is configured to connect with outlet channel 44 and to drive the blood sample from upstream end 14 of patient-specific flow channel 12 to downstream end 16 of patient-specific flow channel 12.

In some embodiments, microvessel-on-chip device 10 can be utilised as a part of a test kit 50, such as that shown in FIG. 3. Test kit 50 includes a portable housing 52 to or in which microvessel-on-chip device 10 can be suitably received. In such an embodiment, the pump may be provided in the housing, and be operatively associated with a suitable actuator (e.g. a push button 56) that when actuated by a user drives the blood sample through patient-specific flow channel 12.

The microvessel-on-chip device 10 enables assessment of Virchow's triad, which describes the three broad categories of factors that are thought to contribute to a blood clot (thrombosis)—hypercoagulability, endothelial dysfunction (endothelial injury) and altered blood flow (stasis of blood flow). In order to evaluate the coagulability, the occlusion time and size of thrombi can be recorded. The location of the thrombi is also documented in relation to the patient-specific altered blood flow. To assess the impact of endothelial dysfunction, a pseudo drug, such as PMA or TNF-a, is mixed with a blood sample to simulate endothelial injury. The change in occlusion time can then be recorded to evaluate the effect. For example, the blood flow condition of a 70-year-old female patient, categorised under Virchow's Triad, was accurately assessed using microvessel-on-chip device 10. This involved precisely 3D fabricating her blood vessel geometry, as detailed in Zhao et al., 2023, Advanced Functional Materials, 33, 1-1-13 (DOI: 10.1002/adfm.202214179), which is incorporated herein by reference. In another example, hypercoagulability is determined not by adjusting the blood condition, but by directly testing the patient's blood using microvessel-on-chip device 10.

It is envisaged that test kit 50 could be used in conjunction with a smart device, such as a smart phone, to send images of the microvessel-on-chip device 10 post-testing for remote assessment. Such an application would reduce the need of a patient to make trips to a dedicated facility in order to undertake the test or to receive results of the test. This integration of tele-health with embodiments of the present invention therefore offers improved accessibility to healthcare services, as patients would be able to receive test results without the need for physical visits to a clinic. The utilisation of smartphone and internet services also ensures high-speed and reliable communication, improving the overall patient experience and outcomes.

With reference to FIG. 4, a method 100 of producing the patient-specific microvessel-on-chip device 10 will now be described below.

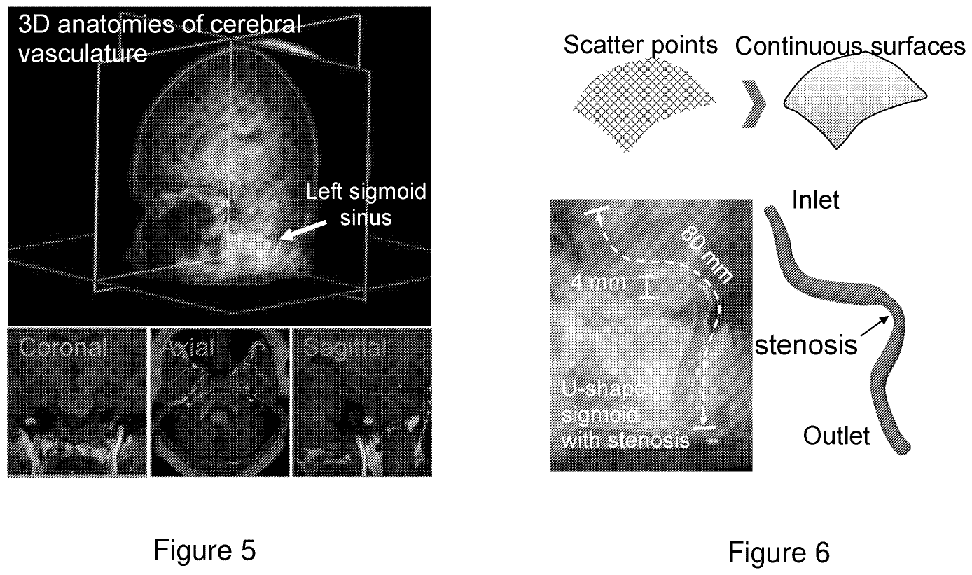

In order to personalise the microvessel-on-chip device 10, it is important to capture and replicate patient specific anatomies. In step 110, clinical images are obtained of a patient's vasculature. The clinical images may be in the form of CT scans, MRI scans (e.g. MRV scans), or any other scans that suitably captures the patient's vasculature. Multiple scans may be captured in order to acquire the necessary 3D information. In one example shown in FIGS. 5, 3D anatomies of cerebral vasculature may be captured such as that of the superior sagittal sinus, sigmoid sinus, and/or the straight sinus in one or more of the coronal, sagittal and axial planes of the patient. In this example, microvessel-on-chip device 10 can be a microfluidic model that mimics the venous geometry of a cerebral venous sinus thrombosis (CVST) patient. It will be appreciated that the more information that is captured and utilised to produce the microvessel-on-chip device 10, the more accurate the model will be, and therefore the more accurate the mimicking of the blood flow through the blood vessel will be.

In step 120, the obtained clinical images are processed in suitable imaging software, and a suitable file is produced of the patient's vasculature for stereolithography (SLA) 3D printing. In the present embodiment, the image processing involves converting the images into continuous 3D vascular contours. Vascular geometries are then reconstructed and smoothed using a suitable computer smoothing algorithm. This generally involves formation of continuous surfaces from the scatter points associated with the vascular geometry. The vascular geometries are scaled down in the software to about 400 μm in diameter to save on materials and cost for the subsequent moulding procedures. However, it will be appreciated that this is only exemplary and the scaling down can be to any suitable level based on material availability, cost constraints, and/or time. FIG. 6 provides an example of a reconstructed sigmoid sinus, derived from the MRI scan, having a constriction (i.e. stenosis) between an inlet and outlet end thereof.

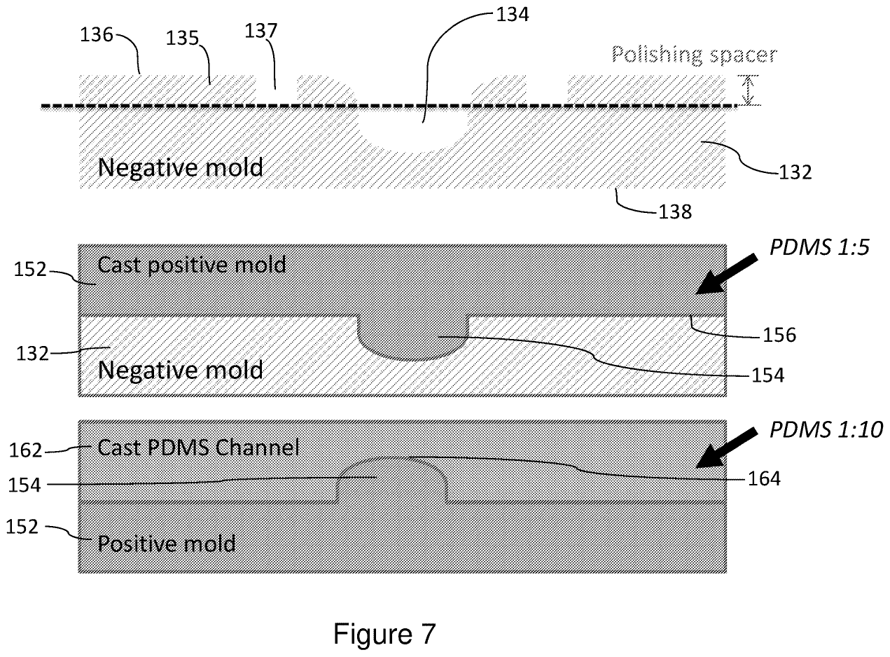

In step 130, the file containing the patient's vasculature is used in a SLA 3D printing process to generate two substantially hemispherical negative master moulds 132 (only one is shown in FIG. 7 for clarity) of the patient's vasculature. The inventors have found that attempts to 3D print the patient's vasculature in a single step results in an inaccurate representation of the vasculature. In particular, the complex shaping of vasculature generally means that it can be very difficult to accurately replicate the intricate surface profile of the vasculature in a single step process. The inventors have found that a desired level of accuracy can be achieved through SLA 3D printing by producing said two substantially hemispherical negative master moulds 132 of the patient's vasculature, with these two hemispherical moulds 132 ultimately utilised in combination to produce the final microvessel-on-chip device 10 in a two-stage moulding process as will be described further below.

In the present embodiment, the two transparent substantially hemispherical negative master moulds 132 are formed from Formlabs Clear Resin (v4), although this is only exemplary and the two substantially hemispherical negative master moulds can be formed of another suitable material for SLA 3D printing.

As best shown in FIG. 7, the formed negative master moulds include an elongate recess 134 having a substantially hemispherical profile when viewed in transverse cross-section that is formed extending from an inner surface 136 of mould 132 towards outer surface 138 thereof. It will be appreciated that whilst the recess is defined as having a substantially hemispherical profile when viewed in transverse cross-section, the recess in fact will have a complex surface profile corresponding to about a half of the interior surface profile of the patient's modelled vasculature. The negative master moulds 132 further includes a polishing spacer 135, which is formed in the negative master mould by forming a substantially rectangular indentation 137 around the recess 134.

At step 140, the inner surface 136 of each of the two negative master moulds 132 are polished in order to smoothen the inner surface 136 of each negative master mould 136. As will be appreciated by a person skilled in the art, a well-known side effect of SLA 3D printing is the effect of light scattering on curved surfaces. As a result, the polishing procedure is desirable to form an accurate negative master mould 132. In the present embodiment, two stages of polishing are undertaken. Firstly, a coarse filing stage is undertaken, such as by using a fine sandpaper. Secondly, a finer filing stage is undertaken, such as by using a cotton wheel (with suitable polishing wax). Polishing of the inner surface 136 is complete when the polishing spacer 135 has been whittled away such that all that remains is a substantially flat inner surface having the recess 134 extending therein (see FIG. 7). Whilst the polishing procedure has been described as being undertaken in two stages, this need not be the case. It is envisaged that the polishing procedure can be undertaken in additional stages, or even in a single stage.

To further assess whether the polishing procedure has been satisfactorily completed, optical sensors or other equipment (e.g. scanning electron microscope) may be utilised to check the flatness of the inner surface 136. A precise tolerance of ±1 μm can be achieved in particular regions of the negative master mould 132.

At step 150, two PDMS (polydimethylsiloxane) chips 152 are cast from the two polished negative master moulds 132 (see FIG. 7). The PDMS chips 152 are cast by introducing a PDMS/curing agent mixture to the negative master moulds, with the mixture bound by a suitable aluminium foil in this example. The PDMS/curing agent mixture may have a PDMS/curing agent ratio between about 1:4 to about 1:6. Preferably, the mixture includes a PDMS/curing agent ratio of about 1:5. Providing the PDMS/curing agent mixture within this ratio range assists in allowing the cast PDMS chips 152 to be peeled away from the negative master mould 132. In some cases, the PDMS chips 152 may also undergo a silane treatment, which acts as a de-moulding agent, to further assist the PDMS chips 152 from being peeled off from the respective negative master moulds 132.

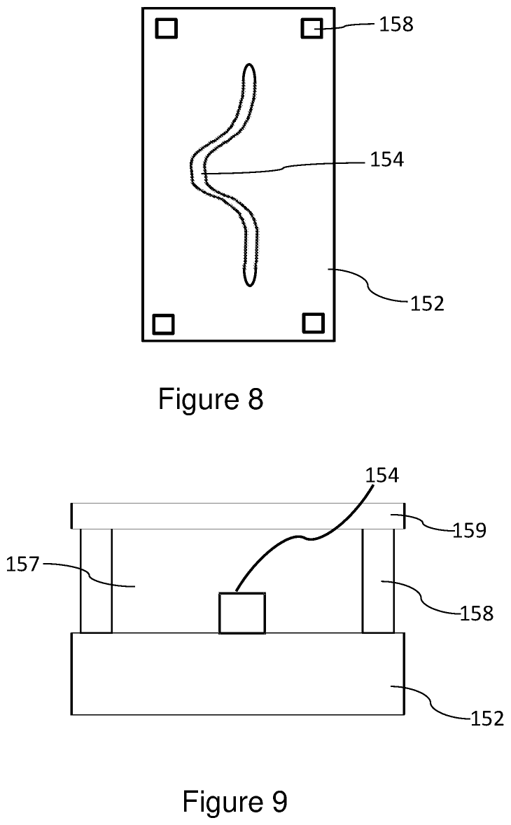

The formed PDMS chips 152 will act as a positive mould for the second stage of the two-stage moulding process. The positive mould PDMS chips 152 each have a substantially hemispherical protrusion 154 that extends from an inner surface 156 of each PDMS chip 152. The protrusion 154 will have a shape that substantially corresponds to that of the recess 134 of the negative master mould 132. The positive mould PDMS chips 152 further include moulding spacers 158 (FIGS. 8 and 9) that project away from the inner surface of the chips 152. The moulding spacers 158 are integrally formed with PDMS chip during the positive mould PDMS chip forming process. However, it will be appreciated that the moulding spacers 158 could be affixed to the positive mould PDMS chips in a separate process. In the present embodiment, the moulding spacers 158 are in the form of column members, wherein one column member is disposed adjacent to each corner of the positive mould PDMS chips 152 (4 columns in total on each chip). However, in other embodiments, the moulding spacers 158 may have a rounded external profile to enhance release of the subsequent moulded portion. The moulding spacers are configured to support a glass cover slide 159 in spaced relation with the positive mould PDMS chip 152 for the subsequent moulding process described below. The moulding spacers 158 can have a length between about 300 μm and 600 μm. Preferably, the moulding spacers have a length of about 170 μm from the inner surface of chips 152.

At step 160, the second stage of the two-stage moulding process is carried out using the positive mould PDMS chips 152. A glass cover slide 159 is supported in spaced relation with the positive mould PDMS chips 152 by resting the glass cover slide 159 on the moulding spacers 158. This creates a moulding chamber 157 between the positive mould PDMS chip 152 and the cover slide 159. PDMS chips 162 are then cast by injection moulding. A PDMS/curing agent mixture is injected into the moulding chamber 157 using a syringe, with the mixture bound laterally. The PDMS/curing agent mixture may have a PDMS/curing agent ratio between about 1:9 and about 1:11. Preferably, the mixture includes a PDMS/curing agent ratio of about 1:10. Once the mixture has cured, two PDMS chips 162 each having a recess 164 defining a negative of the desired vasculature is produced. In some cases, the PDMS chips 162 can undergo a further silane treatment, which again acts as a de-moulding agent, to further assist the PDMS chips 162 from being peeled off from the positive mould PDMS chip 152 and the cover slide 159. The shaping of moulding spacers 158 (as mentioned earlier) can enhance release of PDMS chips 162 from positive mould PDMS chip 152 and the cover slide 159.

At step 170, an inlet channel and an outlet channel is formed in one of the two formed PDMS chips 162. The inlet and outlet channels extend from an outer surface of the PDMS chip 162 to an inner surface of the PDMS chip 162 such that the inlet channel and the outlet channel are in fluid communication with the recess 164. The inlet channel extends through to an upstream end of the recess, whilst the outlet channel extends through to a downstream end of the recess as earlier described with respect to FIG. 2.

At step 180, the PDMS chips undergo a plasma cleaning process to remove any impurities or contaminants from the surfaces of the PDMS chips 162.

At step 190, the two PDMS chips 162 are joined together, in the present embodiment covalently bonded, along the respective inner surfaces of each PDMS chip 162. In the present example, a suitable adhesive is applied between the two PDMS chips (e.g. gelatin methacryloyl—GelMA) and the PDMS chips are then exposed to UV light to complete the bonding process. Once bonded together, the two elongate substantially hemispherical recesses form a whole elongate substantially spherical channel, configured to enable passage of fluid from an upstream end thereof to a downstream end.

At step 200, the formed microvessel-on-chip device 10 undergoes an endothelialisation process to improve the biocompatibility of the microvessel-on-chip device 10. In one example, this process involves introducing about a 20 μL of 5×106 cells/ml of human umbilical vein endothelial cells (HUVECs) suspension in EGM-2 culture medium into the substantially spherical channel via the inlet channel and incubating the microvessel-on-chip device 10 for a period of about 20 minutes. The microvessel-on-chip device 10 is then inverted and maintained in this inverted position for about 20 minutes to allow the HUVECs to attach to the top of the substantially spherical channel. 200 μL of EGM-2 culture medium is added to the substantially spherical channel, which is then placed in an incubator at 37° C. and 5% CO₂ for static overnight culture. Once this has been completed, the endothelialised microvessel-on-chip device 10 is ready for blood clot tests. It is envisaged that the blood sample utilised may be about 200 μL, although this is only exemplary. Thus, microvessel-on-chip device 10 includes living endothelial functionalization and whole blood perfusion to recapitulate the patient-specific Virchow's triad.

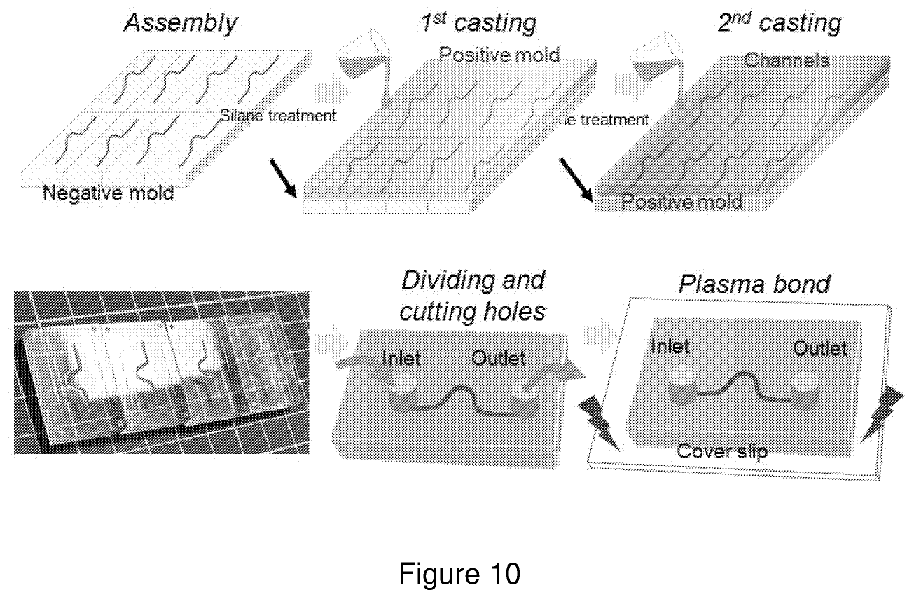

Whilst the above method has been described in relation to producing a single microvessel-on-chip device, various steps of the process lend themselves to mass producing a plurality of microvessel-on-chip devices. Reference is made to FIG. 10, which provides an illustration of some steps in the manufacturing process that can be scaled up to provide production of microvessel-on-chip devices at scale. In particular, the two-stage moulding process (steps 150, 160) can be undertaken in bulk by first assembling various negative master moulds in a suitable arrangement. For example, the negative master moulds can be arranged in suitable columns and rows, wherein a particular column may denote negative moulds for a particular patient, and particular rows denote negative moulds for a particular vessel belonging to a given patient. In other embodiments, a single negative master mould may be formed by the SLA 3D printing process having the plurality of recesses suitably spaced along the negative master mould.

The first stage of the moulding process can then be undertaken by casting the plurality of PDMS chips by introducing the PDMS/curing agent mixture to the assembly of negative master moulds. This results in a larger PDMS slab being produced with each of the individual substantially hemispherical protrusions extending from an inner surface of the slab. The positive mould PDMS slab may then undergo a silane treatment to assist the positive mould PDMS slab from being peeled off from the assembled negative master moulds. The second stage of the moulding process can then be undertaken using the positive mould PDMS slab. This will involve casting the plurality of PDMS chips by introducing the PDMS/curing agent mixture to a moulding chamber of the positive mould PDMS slab. Again, suitable moulding spacers may be utilised, either integrally formed with the positive mould PDMS slab, or affixed to the positive mould PDMS slab in a separate process. This results in another PDMS slab being produced having the individual substantially hemispherical recesses extending from an inner surface of the slab towards an outer surface of the PDMS slab.

The PDMS slab having the substantially hemispherical recesses can now be suitably divided into individual chips through a suitable dividing or cutting process, and the inlet and outlet channels can be suitably formed in one of the two microvessel-on-chip device portions before the portions are joined together to form the final microvessel-on-chip device.

It is envisaged that large-scale production of multiple microvessel-on-chip devices will significantly reduce fabrication time and cost.

Thus, the microvessel-on-chip device disclosed herein can be used to recapitulate a patient's Virchow's triad. With this device, Virchow's triad can be assessed in a number of ways. Blood flow can be monitored by filling the flow channel with fluorescent particles or the patient's blood sample, and the haemodynamics (blood flow pattern) can be monitored using existing micro-PIV techniques that are widely available. Endothelial dysfunction can be monitored using the device in conjunction with inflammatory drugs or physical modalities that damage the endothelial cells, with the response of blood clots to endothelial damage monitored under a camera. The coagulability of the patient's blood can be assessed by observing the formation of blood clots using a camera or microscope, based on the presence of increased coagulation factors or calcium in the blood plasma.

It will be understood that the invention disclosed and defined in this specification extends to all alternative combinations of two or more of the individual features mentioned or evident from the text or drawings. All of these different combinations constitute various alternative aspects of the invention.

1. A method of manufacturing a patient-specific microfluidic device, the method including:
obtaining three-dimensional (3D) information associated with vascular geometry in a region of interest from one or more clinical images associated with a patient;
fabricating the patient-specific microfluidic device using said 3D information, wherein said patient-specific microfluidic device includes a flow channel having a geometry that substantially corresponds to the vascular geometry in the region of interest.

2. The method of claim 1, wherein the one or more clinical images include one or more of magnetic resonance imaging (MRI) scans, computerised tomography (CT) scans, and any other clinical images that contain 3D anatomical information.

3. The method of claim 1 or 2, wherein said obtaining 3D information includes identifying the region of interest from the one or more clinical images, and converting the one or more clinical images into 3D vascular contours at least in the region of interest.

4. The method of any one of the preceding claims, wherein the 3D vascular contours are generated from a plurality of scatter points associated with the vascular geometry in the region of interest, and wherein the 3D vascular contours include continuous surfaces constructed from the scatter points, thereby producing surface profiles of the vascular geometry in the region of interest.

5. The method of claim 4, wherein the surface profiles of the vascular geometry in the region of interest are smoothed using a computer algorithm.

6. The method of any one of the preceding claims, wherein the method further includes fabricating a mould from said 3D information, wherein said mould includes geometry that substantially corresponds to the vascular geometry in the region of interest.

7. The method of claim 6, wherein said fabricating a mould includes fabricating a first mould portion and a second mould portion, wherein said first mould portion includes geometry that substantially corresponds to a first portion of the vascular geometry in the region of interest, and said second mould portion includes geometry that substantially corresponds to a second portion of the vascular geometry in the region of interest, wherein the first portion and the second portion together define geometry that substantially corresponds to the entirety of the vascular geometry in the region of interest.

8. The method of claim 7 or 8, wherein said geometry of the first mould portion that substantially corresponds to the first portion of the vascular geometry in the region of interest is in the form of a recess extending from an inner surface of the first mould portion towards an outer surface of the first mould portion, and said geometry of the second mould portion that substantially corresponds to the second portion of the vascular geometry in the region of interest is in the form of a recess extending from an inner surface of the second mould portion towards an outer surface of the second mould portion, wherein each of the respective recesses of the first mould portion and the second mould portion include a substantially hemispherical internal profile generally corresponding to the respective first and second portions of the vascular geometry in the region of interest.

9. The method of any one of the preceding claims, wherein said fabricating the mould includes fabricating the mould by an additive manufacturing process, preferably using a stereolithography (SLA) 3D printing process.

10. The method of claim 8 or claim 9 insofar as dependent thereon, wherein fabricating the first mould portion includes forming a groove extending from the inner surface of the first mould portion towards the outer surface of the first mould portion, wherein said groove substantially surrounds the recess, wherein said groove at least in part delineates a spacer that substantially surrounds the recess, wherein a height of said spacer corresponds to a depth of the groove and defines a portion of the first mould portion to be removed, and
wherein fabricating the second mould portion includes forming a groove extending from the inner surface of the second mould portion towards the outer surface of the second mould portion, wherein said groove substantially surrounds the recess, wherein said groove at least in part delineates a spacer that substantially surrounds the recess, wherein a height of said spacer corresponds to a depth of the groove and defines a portion of the second mould portion to be removed.

11. The method of claim 7 or any one of claims 8 to 11 insofar as dependent thereon, further including fabricating a secondary mould from the mould, wherein said secondary mould includes geometry that substantially corresponds to the vascular geometry in the region of interest.

12. The method of claim 11, wherein fabricating the secondary mould includes fabricating a first secondary mould portion and a second secondary mould portion, wherein said first secondary mould portion includes geometry that substantially corresponds to the first portion of the vascular geometry in the region of interest, and said second secondary mould portion includes geometry that substantially corresponds to the second portion of the vascular geometry in the region of interest, and wherein the method further includes fabricating the first secondary mould from the first mould portion and fabricating the second secondary mould from the second mould portion.

13. The method of claim 12, wherein said geometry of the first secondary mould portion that substantially corresponds to the first portion of the vascular geometry in the region of interest is in the form of a protrusion extending outwardly from an inner surface of the first secondary mould portion, and wherein said geometry of the second secondary mould portion that substantially corresponds to the second portion of the vascular geometry in the region of interest is in the form of a protrusion extending outwardly from an inner surface of the second secondary mould portion, wherein each of the respective protrusions of the first secondary mould portion and the second secondary mould portion include a substantially hemispherical external profile corresponding to the respective first and second portions of the vascular geometry in the region of interest.

14. The method of any one of claims 11 to 13, wherein the secondary mould is formed at least in part of polydimethylsiloxane (PDMS), wherein preferably the secondary mould is formed of a PDMS/curing agent mixture having a PDMS/curing agent ratio between about 1:4 to about 1:6.

15. The method of claim 12 or any one of claims 12 to 14 insofar as dependent thereon, wherein the first secondary mould portion includes one or more moulding spacers projecting from an inner surface thereof, and/or wherein the second secondary mould portion includes one or more moulding spacers projecting from an inner surface thereof,
wherein the one or more moulding spacers are configured to support a glass cover slide in spaced relation from the inner surface of the respective first and/or second secondary mould portion.

16. The method of any one of claims 16 to 23, wherein fabricating the patient-specific microfluidic device includes forming the patient-specific microfluidic device from the secondary mould, wherein said patient-specific microfluidic device includes geometry that substantially corresponds to the vascular geometry in the region of interest.

17. The method of claim 7 or any one of claims 8 to 16 insofar as dependent thereon, wherein the patient-specific microfluidic device includes a first patient-specific microfluidic device portion and a second patient-specific microfluidic device portion, wherein said first patient-specific microfluidic device portion includes geometry that substantially corresponds to the first portion of the vascular geometry in the region of interest, and said second patient-specific microfluidic device portion includes geometry that substantially corresponds to the second portion of the vascular geometry in the region of interest, and
wherein said geometry of the first patient-specific microfluidic device portion that substantially corresponds to the first portion of the vascular geometry in the region of interest is in the form of a recess extending from an inner surface of the first patient-specific microfluidic device portion towards an outer surface of the first patient-specific microfluidic device portion, and wherein said geometry of the second patient-specific microfluidic device portion that substantially corresponds to the second portion of the vascular geometry in the region of interest is in the form of a recess extending from an inner surface of the second patient-specific microfluidic device portion to an outer surface of the second patient-specific microfluidic device portion.

18. The method of claim 17 insofar as dependent on claim 12, further including fabricating the first patient-specific microfluidic device portion from the first secondary mould portion and forming the second patient-specific microfluidic device portion from the second secondary mould portion.

19. The method of claim 17 or 18, insofar as dependent on claim 15, wherein said forming the first patient-specific microfluidic device portion and the second patient-specific microfluidic device portion includes positioning a glass cover slide on the one or more moulding spacers of the respective first and second secondary mould portions such that the glass cover is in spaced relation with the inner surface of the respective first and second secondary mould portions, wherein a moulding chamber is formed between the glass cover and the inner surface of the respective first and second secondary mould portion, and
the method further includes injecting a settable material into the respective moulding chambers, allowing the settable material to cure, thereby forming said first and second patient-specific microfluidic device portion.

20. The method of claim 19, wherein the settable material is a PDMS/curing agent mixture, wherein preferably the PDMS/curing agent mixture has a PDMS/curing agent ratio between about 1:9 to about 1:11.

21. The method of any one of the preceding claims, further including forming an inlet channel and an outlet channel in the patient-specific microfluidic device, wherein the inlet channel and the outlet channel are in fluid communication with the flow channel.

22. The method of claim 21 or any one of claims 19 to 20 insofar as dependent thereon, further including joining the first patient-specific microfluidic device portion to the second patient-specific microfluidic device portion along the respective inner surfaces thereof, such that the recess of each respective microfluidic device portion substantially overlap along their longitudinal extent, thereby forming said flow channel having the geometry that substantially corresponds to the vascular geometry in the region of interest.

23. The method of any one of the preceding claims, further including endothelialising the patient-specific microfluidic device.

24. A patient-specific microfluidic device manufactured using the method of any one of claims 1 to 23.

25. A patient-specific microfluidic device, the device including a flow channel having a geometry that substantially corresponds to a patient's vascular geometry in a region of interest, said geometry derived from one or more clinical images associated with the patient.

26. The device of claim 25, wherein the one or more clinical images include one or more of magnetic resonance imaging (MRI) scans, computerised tomography (CT) scans, and any other clinical images that contain 3D anatomical information.

27. The device of claim 25 or 26, wherein the patient-specific microfluidic device includes a first patient-specific microfluidic device portion and a second patient-specific microfluidic device portion joined to the first patient-specific microfluidic device portion, wherein said first patient-specific microfluidic device portion includes geometry that substantially corresponds to a first portion of the vascular geometry in the region of interest, and said second patient-specific microfluidic device portion includes geometry that substantially corresponds to a second portion of the vascular geometry in the region of interest, wherein the first portion and the second portion together define geometry that substantially corresponds to the entirety of the vascular geometry in the region of interest.

28. The device of claim 27, wherein the geometry of the first patient-specific microfluidic device portion that substantially corresponds to the first portion of the vascular geometry in the region of interest is in the form of a recess extending from an inner surface of the first patient-specific microfluidic device portion towards an outer surface of the first patient-specific microfluidic device portion, and wherein the geometry of the second patient-specific microfluidic device portion that substantially corresponds to the second portion of the vascular geometry in the region of interest is in the form of a recess extending from an inner surface of the second patient-specific microfluidic device portion to an outer surface of the second patient-specific microfluidic device portion, wherein each of the respective recesses of the first patient-specific microfluidic device portion and the second patient-specific microfluidic device portion may include a substantially hemispherical internal profile generally corresponding to the respective first and second portions of the vascular geometry in the region of interest.

29. The device of claim 28, wherein the first patient-specific microfluidic device portion and the second patient-specific microfluidic device portion are joined along respective inner surfaces thereof, such that the recess of each respective microfluidic device portion substantially overlap along their longitudinal extent, thereby forming said flow channel having the geometry that substantially corresponds to the vascular geometry in the region of interest.

30. The device of any one of claims 25 to 29, wherein the patient-specific microfluidic device includes an inlet channel in fluid communication with an upstream end of the flow channel, and an outlet channel in fluid communication with a downstream end of the flow channel; and/or
wherein the patient-specific microfluidic device includes endothelial tissue, said endothelial tissue covering at least a portion of the flow channel, wherein the endothelial tissue covers at least a portion of a wall, which at least in part defines said flow channel.
